(19) United States
(12) Patent Application Publication
(10) Pub. No.: US 2023/0174455 A1
(43) Pub. Date: Jun. 8, 2023

(54) RECYCLED CONTENT TRIACETIN

(71) Applicant: Eastman Chemical Company, Kingsport, TN (US)

(72) Inventors: Kenneth James Flint, Roanoke, VA (US); Michael Neil Brown, Kingsport, TN (US); Hannah Grace Moser, Gate City, VA (US); Evan Patrick Beaumier, Kingsport, TN (US)

(73) Assignee: Eastman Chemical Company, Kingsport, TN (US)

(21) Appl. No.: 18/052,270

(22) Filed: Nov. 3, 2022

Related U.S. Application Data
(63) Continuation-in-part of application No. PCT/US2022/025663, filed on Apr. 21, 2022.
(60) Provisional application No. 63/201,576, filed on May 5, 2021.

(51) Int. Cl. C07C 67/12; C10J 3/84; C07C 29/151; C07C 67/08; C07C 51/56; C07C 51/12

(52) U.S. Cl. CPC C07C 67/12; C10J 3/84; C07C 29/1518; C07C 67/08; C07C 51/56; C07C 51/12; C10J 2300/0946; C10J 2300/1665

(57) ABSTRACT

Recycled content triacetin (r-triacetin) is produced using a process and system that applies physical and/or credit-based recycled content from one or more feed materials to triacetin produced from the feed materials.

### CROSS REFERENCES TO RELATED APPLICATIONS

This application is a Continuation-in-Part application of PCT Application Number PCT/US2022/025663, filed Apr. 21, 2022, which claims priority to U.S. Provisional Application No. 63/201,576, filed May 5, 2021.

### BACKGROUND

Triacetin is commonly used as a plasticizer for cellulosic resins and is compatible in all proportions with cellulose acetate, nitrocellulose, and ethyl cellulose. Triacetin is useful for imparting plasticity and flow to laminating resins, particularly at low temperatures, and is also used as a plasticizer for vinylidene polymers and copolymers. Triacetin also serves as an ingredient in inks for printing on plastics, and as a plasticizer in nail polish.

The demand for recycled chemical products continues to grow, but there is no clear path to recycled triacetin through mechanical recycling. Thus, there exists a need for a commercial process to produce recycled triacetin.

### SUMMARY

In one aspect, the present technology concerns a process for producing triacetin having recycled content, where the process comprises the following steps: (a) carbon reforming a feed comprising waste plastic to thereby produce a recycled content syngas (r-syngas); (b) converting at least a portion of the r-syngas to recycled content methanol (r-methanol) via catalytic synthesis; (c) producing a recycled content acetic anhydride (r-acetic anhydride) from at least a portion of the r-methanol and a carbon monoxide (CO); and (d) acetylating a glycerin with at least a portion of the r-acetic anhydride to thereby provide the r-triacetin.

In one aspect, the present technology concerns a process for producing triacetin having recycled content, where the process comprises the following steps: (a) converting a syngas to a methanol via catalytic synthesis; (b) producing an acetic anhydride from at least a portion of the methanol and a carbon monoxide (CO); (c) acetylating a glycerin with at least a portion of the acetic anhydride to thereby provide a triacetin; and (d) applying recycled content to at least a portion of the triacetin to thereby provide a recycled content triacetin (r-triacetin). The applying of step (d) includes (i) attributing recycled content from at least one source material having physical recycled content to at least one target material via recycled content credits, (ii) tracing recycled content along at least one chemical pathway from the at least one target material to the triacetin, and (iii) allocating recycled content to the triacetin based at least in part on the tracing of recycled content along the chemical pathway.

In one aspect, the present technology concerns a process for producing triacetin having recycled content (r-triacetin), the process comprising: (a) carbon reforming a feed comprising waste plastic to thereby produce a recycled content syngas (r-syngas); (b) converting at least a portion of the r-syngas to recycled content methanol (r-methanol) via catalytic synthesis; (c) carbonylating at least a portion of the r-methanol with a carbon monoxide (CO) to form recycled content acetic acid (r-acetic acid); and (d) esterifying a glycerin with at least a portion of the r-acetic acid to thereby provide the r-triacetin.

In another aspect, the present technology concerns a process for producing triacetin having recycled content (r-triacetin), the process comprising: (a) carbon reforming a feed comprising waste plastic to thereby produce a recycled content syngas (r-syngas); (b) converting at least a portion of the r-syngas to recycled content methanol (r-methanol) via catalytic synthesis; (c) producing a recycled content acetic anhydride (r-acetic anhydride) from at least a portion of the r-methanol and a carbon monoxide (CO); (d) recovering a recycled content acetic acid (r-acetic acid) from an acetylation with at least a portion of the r-acetic anhydride; and (e) esterifying a glycerin with at least a portion of the r-acetic acid to thereby provide the r-triacetin.

In another aspect, the present technology concerns a process for producing triacetin having recycled content (r-triacetin), the process comprising: (a) converting a syngas to a methanol via catalytic synthesis; (b) producing an acetic acid from at least a portion of the methanol and a carbon monoxide (CO); (c) esterifying a glycerin with at least a portion of the acetic acid to thereby provide a triacetin; and (d) applying recycled content to at least a portion of the triacetin to thereby provide the r-triacetin, wherein the applying of step (d) includes (i) attributing recycled content from at least one source material having physical recycled content to at least one target material via recycled content credits, (ii) tracing recycled content along at least one chemical pathway from the at least one target material to the triacetin, and (iii) allocating recycled content to the triacetin based at least in part on the tracing of recycled content along the chemical pathway.

### BRIEF DESCRIPTION OF THE DRAWINGS

FIG. 1 is a block flow diagram illustrating the main steps of a process and facility for making recycled content triacetin (r-triacetin), where the r-triacetin has physical recycled content from waste plastic via recycled content syngas (r-syngas);

FIG. 2 is a block flow diagram illustrating the main steps of an alternative process and facility for making r-triacetin having physical recycled content from waste plastic via r-syngas, but differing from the embodiment of FIG. 1 in that the esterification step is eliminated and a dehydration step is added;

FIG. 3 is a block flow diagram illustrating the main steps of a process and facility for making r-triacetin, where the r-triacetin has physical recycled content from waste plastic via r-syngas and recycled content carbon monoxide (r-CO);

FIG. 4 is a block flow diagram illustrating the main steps of a process and facility for making r-triacetin, where the r-triacetin has credit-based recycled content from r-syngas and r-CO;

FIG. 5 is a block flow diagram illustrating the main steps of a process and facility for making r-triacetin, where the r-triacetin has physical recycled content from r-syngas and credit-based recycled content from r-CO;

FIG. 6 is a block flow diagram illustrating the main steps of a process and facility for making r-triacetin, where the r-triacetin has credit-based recycled content from r-syngas and physical recycled content from r-CO;

FIG. 7 is a block flow diagram illustrating the main steps of an alternative process and facility for making r-triacetin having physical recycled content from waste plastic via r-syngas (and optionally r-carbon monoxide), but differing from the embodiments of FIGS. 1 and 2 in that glycerin is esterified with acetic acid to form the r-triacetin;

FIG. 8 is a block flow diagram illustrating the main steps of a process and facility for making r-triacetin similar to the embodiment illustrated in FIG. 1, but esterifying a glycerin with at least a portion of the r-acetic acid to form an additional stream of r-triacetin;

FIG. 9 is a block flow diagram illustrating the main steps of a process and facility for making r-triacetin similar to the alternative embodiment illustrated in FIG. 2, but esterifying a glycerin with at least a portion of the r-acetic acid to form an additional stream of r-triacetin; and

FIG. 10 is a block flow diagram illustrating the main steps of a process and facility for making r-triacetin, where the r-triacetin has credit-based and/or physical-based recycled content from r-syngas and/or r-CO.

### DETAILED DESCRIPTION

We have discovered new methods and systems for producing triacetin having recycled content. More specifically, we have discovered a process and system for producing triacetin where recycled content from waste materials, such as waste plastic, are applied to triacetin in a manner that promotes the recycling of waste plastic and provides triacetin with substantial amounts of recycled content.

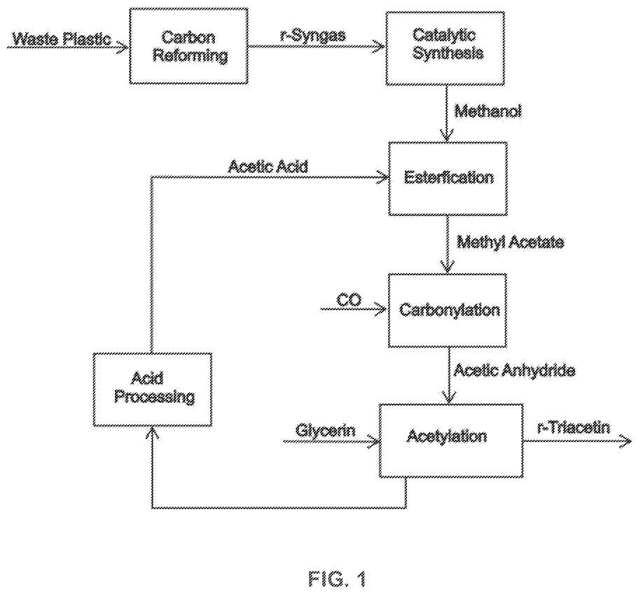

FIG. 1 depicts a process and facility for producing recycled content triacetin (r-triacetin) in accordance with one embodiment of the present technology. As shown in FIG. 1 a waste material, such as waste plastic, can be subjected to carbon reforming to produce a recycled content syngas (r-syngas) having physical recycled content. In one embodiment, the feed to carbon reforming can comprise both a recycled content feed component (e.g., waste plastic) and a non-recycled content feed component (e.g., coal, a liquid hydrocarbon, and/or a gaseous hydrocarbon). In one embodiment, the carbon reforming is partial oxidation gasification that is fed with coal and waste plastic. In another embodiment, the carbon reforming is plasma gasification of a predominately waste plastic feed. In yet another embodiment, the carbon reforming is partial oxidation gasification fed with a non-recycled content liquid or gaseous hydrocarbon and a recycled content pyrolysis oil produced from the pyrolysis of waste plastic.

In the embodiment of FIG. 1, the r-syngas from carbon reforming is fed to a catalytic synthesis step to produce methanol. The methanol is then fed to an esterification step where it is used to esterify acetic acid and produce methyl acetate. The methyl acetate is then fed to a carbonylation step for carbonylation with carbon monoxide (CO) to produce acetic anhydride. The acetic anhydride is then used in an acetylation step to acetylate glycerin and produce the r-triacetin. The acetylation step also produces acetic acid that can be sent to an acid processing step, where it is cleaned up and or combined with added acetic acid and then used in the esterification step.

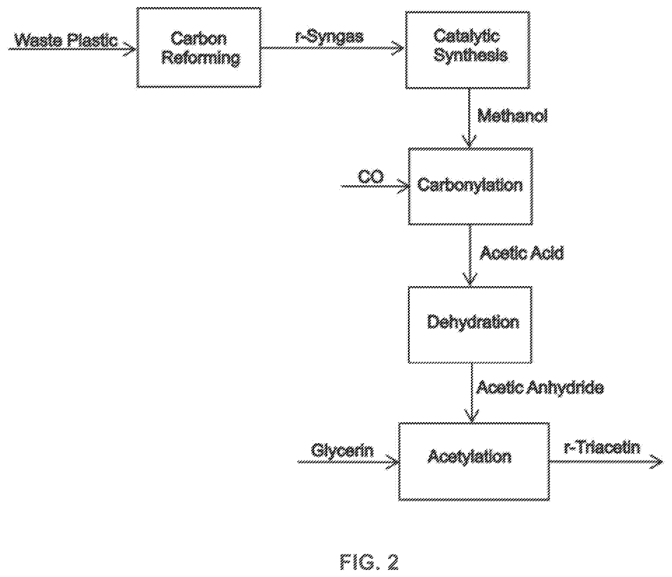

FIG. 2 depicts an alternative triacetin production process and facility that eliminates the esterification step shown in FIG. 1 and adds a dehydration step to prior to acetylation. More specifically, the process depicted in FIG. 2 subjects the methanol from catalytic synthesis to carbonylation with CO to thereby produce acetic acid. The acetic acid from carbonylation can then be dehydrated to acetic anhydride, which can then be used to acetylate glycerin and produce the r-triacetin.

In the embodiments depicted in FIGS. 1 and 2, all the recycled content in the r-triacetin product is physical recycled content that is physically traceable back to the waste plastic. In one or more embodiments, including the embodiments of FIGS. 1 and 2, the r-triacetin has a physical recycled content of 10 to 60, 20 to 50, or 25 to 40 percent, all originating from the waste plastic.

The amount of physical recycled content in the r-triacetin can determined by tracing the amount of recycled material along a chemical pathway starting with waste plastic and ending with the triacetin. The chemical pathway includes all chemical reactions and other processing steps (e.g., separations) between the starting material (e.g., waste plastic) and the triacetin. In FIG. 1, the chemical pathway includes carbon reforming, catalytic synthesis, esterification, carbonylation, and acetylation. In FIG. 2, the chemical pathway includes carbon reforming, catalytic synthesis, carbonylation, dehydration, and acetylation.

In one or more embodiments, a conversion factor can be associated with each step along the chemical pathway. The conversion factors account for the amount of the recycled content diverted or lost at each step along the chemical pathway. For example, the conversion factors can account for the conversion, yield, and/or selectivity of the chemical reactions along the chemical pathway.

The amount of recycled content applied to the r-triacetin can be determined using one of variety of methods for quantifying, tracking, and allocating recycled content among various materials in various processes. One suitable method, known as “mass balance,” quantifies, tracks, and allocates recycled content based on the mass of the recycled content in the process. In certain embodiments, the method of quantifying, tracking, and allocating recycled content is overseen by a certification entity that confirms the accuracy of the method and provides certification for the application of recycled content to the r-triacetin.

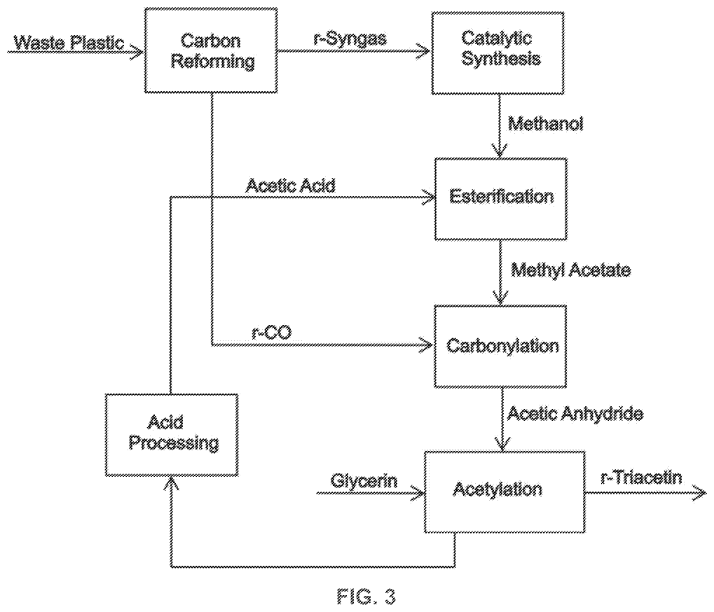

FIG. 3 illustrates an embodiment where both the r-syngas fed to catalytic synthesis and the r-CO fed to carbonylation have physical recycled content from carbon reforming of waste plastic. In one or more embodiments, including the embodiment of FIG. 3, the r-triacetin can have a physical recycled content of 25 to 90, 40 to 80, or 55 to 65 percent. In certain embodiments, the r-triacetin can have 10 to 60, 20 to 50, or 25 to 40 percent physical recycled content from the r-syngas and 10 to 60, 20 to 50, or 25 to 40 percent physical recycled content from the r-CO.

FIGS. 3 through 6 each show a process and facility where the main steps (i.e., carbon reforming, catalytic synthesis, esterification, carbonylation, and acetylation) are the same as the main steps shown in the embodiment of FIG. 1. It should be understood however, that the unique features illustrated in FIGS. 3 through 6 can also be applied to the process shown in FIG. 2, where the main steps are carbon reforming, catalytic synthesis, carbonylation, dehydration, and acetylation.

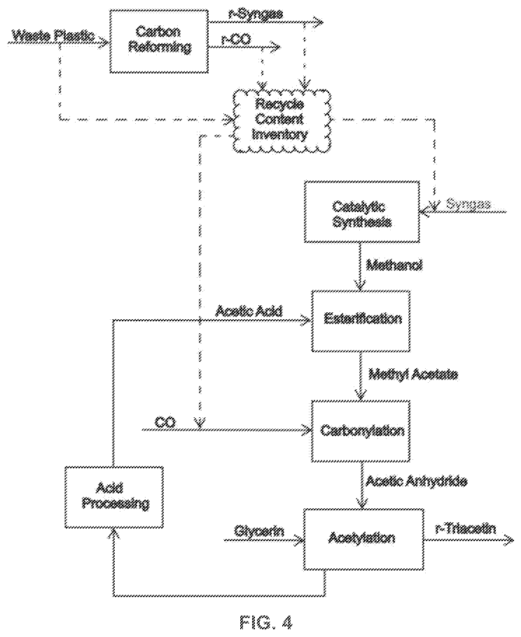

FIG. 4 illustrates an embodiment where the r-triacetin has no physical recycled content but has credit-based recycled content. In the process and system depicted in FIG. 4, the r-syngas produced by carbon reforming of waste plastic is not directly fed to catalytic synthesis. Nor is the r-CO produced by carbon reforming directly fed to carbonylation. Rather, recycled content credits from the r-syngas and r-CO product of carbon reforming are attributed to the syngas and CO fed to catalytic synthesis and carbonylation, respectively. As such, the r-syngas from carbon reforming acts as a “source material” of recycled content credits and the syngas fed to catalytic synthesis acts as a “target material” to which the recycled content credits are attributed. Similarly, the r-CO from carbon reforming acts as a “source material” of recycled content credits and the CO fed to carbonylation acts as a “target material” to which the recycled content credits are attributed.

In one or more embodiments, the source material has physical recycled content and the target material has less than 100 percent physical recycled content. For example, the source material can have at least 10, 25, 50, 75, 90, 99, or 100 percent physical recycled content and/or the target material can have less than 100, 99, 90, 75, 50, 25, 10, or 1 percent physical recycled content.

The ability to attribute recycled content credits from a source material to a target material removes the co-location requirement for the facility making the source material (with physical recycled content) and the facility making the triacetin. This allows a chemical recycling facility/site in one location to process waste material into one or more recycled content source materials and then apply recycled content credits from those source materials to one or more target materials being processed in existing commercial facilities located remotely from the chemical recycling facility/site. Further, the use of recycled content credits allows different entities to produce the source material and the r-triacetin. This allows efficient use of existing commercial assets to produce r-triacetin. In one or more embodiments, the source material is made at a facility/site that is at least 0.1, 0.5, 1, 5, 10, 50, 100, 500, or 1000 miles from the facility/site where the target material is used to make triacetin.

The attributing of recycled content credits from the source material (e.g., the r-syngas product from carbon reforming) to the target material (e.g., the syngas fed to catalytic synthesis) can be accomplished by transferring recycled content credits directly from the source material to the target material. Alternatively, as shown in FIG. 4, recycled content credits can be applied from any of the waste plastic, the r-syngas, and/or the r-CO to the triacetin via a recycled content inventory. The recycled content inventory can be a digital inventory or database used to record and track recycled content for various materials at various sites over various time periods.

When a recycled content inventory is used, recycled content credits from the source material having physical recycled content (e.g., the waste plastic, the r-syngas, and/or the r-CO in FIG. 4) are booked into the recycled content inventory. The recycled content inventory can also contain recycled content credits from other sources and from other time periods. In one embodiment, recycled content credits in the recycled content inventory can only be assigned to target materials having the same or similar composition as the source materials. For example, as shown in FIG. 4, recycled content credits booked into the recycled content inventory from the r-syngas from carbon reforming can be assigned to the syngas fed to catalytic synthesis because the two syngas have the same or similar compositions. However, recycled content credits from r-syngas could not be assigned to the glycerin fed to acetylation because the source and target materials would not be the same or similar.

Once recycled content credits have been attributed to the target material (e.g., the syngas fed to catalytic synthesis and the CO fed to carbonylation in FIG. 4), the amount of the credit-based recycled content allocated to the triacetin from the syngas is calculated by tracing the recycled content along the chemical pathway from the target material (e.g., the syngas and the CO in FIG. 4) to the triacetin. The chemical pathway includes all chemical reactions and other processing steps (e.g., separations) between the target material and the triacetin, and a conversion factor can be associated with each step along the chemical pathway of the credit-based recycled content. The conversion factors account for the amount of the recycled content diverted or lost at each step along the chemical pathway. For example, the conversion factors can account for the conversion, yield, and/or selectivity of the chemical reactions along the chemical pathway.

As with the physical recycled content, the amount of credit-based recycled content applied to the r-triacetin can be determined using one of variety of methods, such as mass balance, for quantifying, tracking, and allocating recycled content among various materials in various processes. In certain embodiments the method of quantifying, tracking, and allocating recycled content is overseen by a certification entity that confirms the accuracy of the method and provides certification for the application of recycled content to the r-triacetin.

The r-triacetin produced by the process of FIG. 4 can have 25 to 90, 40 to 80, or 55 to 65 percent credit-based recycled content and less than 50, 25, 10, 5, or 1 percent physical recycled content. In certain embodiments, the triacetin can have 10 to 60, 20 to 50, or 25 to 40 percent credit-based recycled content from the r-syngas and 10 to 60, 20 to 50, or 25 to 40 percent credit-based recycled content from the r-CO.

In one or more embodiments, the recycled content of the r-triacetin product can include both physical recycled content and credit-based recycled content. For example, the r-triacetin can have at least 10, 20, 30, 40, 50 percent physical recycled content and at least 10, 20, 30, 40, or 50 percent credit-based recycled content. As used herein, the term “total recycled content” refers to the cumulative amount of physical recycled content and credit-based recycled content from all sources.

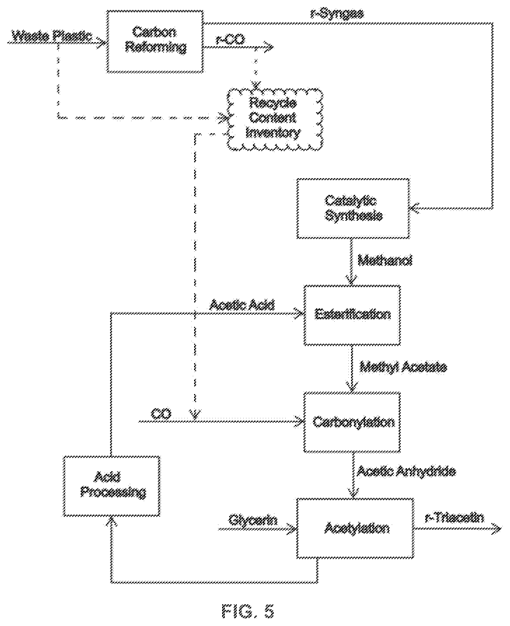

FIG. 5 illustrates a r-triacetin production process and system wherein physical recycled content is supplied via the r-syngas fed to catalytic synthesis and credit-based recycled content is supplied via the CO fed to carbonylation.

In the embodiment depicted in FIG. 5, the r-triacetin product can have a total recycled content of 25 to 90, 40 to 80, or 55 to 65 percent if, for example, (i) the r-syngas fed to catalytic synthesis has 100 percent physical recycled content and (ii) the CO fed to carbonylation has 100 percent credit-based recycled content. In such a scenario, the r-triacetin can have both physical recycled content and credit-based recycled content, including, for example, (i) 10 to 60, 20 to 50, or 25 to 40 percent physical recycled content from the r-syngas and (ii) 10 to 60, 20 to 50, or 25 to 40 percent credit-based recycled content from the r-CO. The amount of recycled content applied to the triacetin product can be varied depending on the physical recycled content and/or credit-based recycled content of the syngas and CO fed into the triacetin production process.

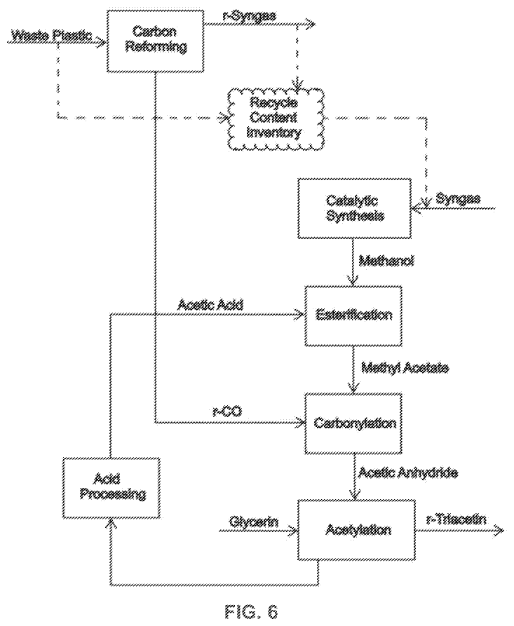

FIG. 6 illustrates a r-triacetin production process and system wherein physical recycled content is supplied via the r-CO fed to carbonylation and credit-based recycled content is supplied via the syngas fed to catalytic synthesis.

In the embodiment depicted in FIG. 6, the r-triacetin product can have a total recycled content of 25 to 90, 40 to 80, or 55 to 65 percent if, for example, (i) the syngas fed to catalytic synthesis has 100 percent credit-based recycled content and (ii) the r-CO fed to carbonylation has 100 percent physical recycled content. In such a scenario, the r-triacetin can have both physical recycled content and credit-based recycled content, including, for example, (i) 10 to 60, 20 to 50, or 25 to 40 percent credit-based recycled content from the r-syngas and (ii) 10 to 60, 20 to 50, or 25 to 40 percent physical recycled content from the r-CO.

In the embodiments depicted in FIGS. 1-3, the carbon reforming facility can be co-located with the r-triacetin production facility. In the embodiments depicted in FIGS. 4-6, the carbon reforming facility can be located remotely from r-triacetin production facility.

Although not illustrated in the drawings, if glycerin having 100 percent recycled content is provided, it enables the production of r-triacetin having 100 percent recycled content, so long as the other feeds (i.e., syngas and CO) also have 100 percent recycled content. In such a scenario, the 100 percent recycled content r-triacetin can have, for example, (i) 10 to 60, 20 to 50, or 25 to 40 percent recycled content (physical and/or credit-based) from the r-syngas, (ii) 10 to 60, 20 to 50, or 25 to 40 percent recycled content (physical and/or credit-based) from the r-CO, and (iii) 15 to 65, 25 to 55, 35 to 45 recycled content (physical and/or credit-based) from r-glycerin.

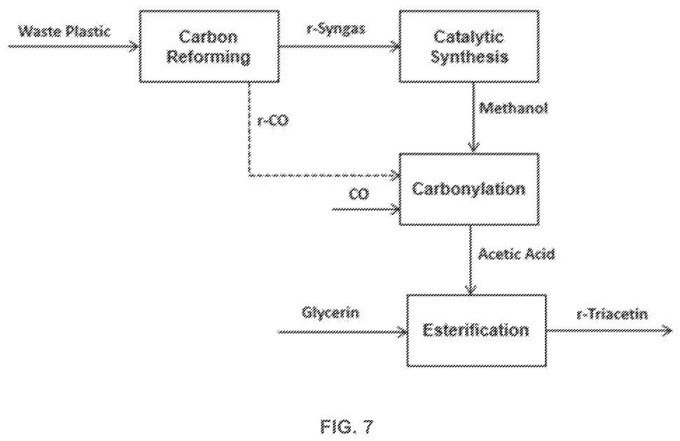

FIG. 7 depicts yet another triacetin production process and facility that eliminates the dehydration step shown in FIG. 2 and directly uses the recycled content acetic acid to form r-triacetin. More specifically, the process depicted in FIG. 7 esterifies a glycerin with at least a portion of the acetic acid from the carbonylation step to produce r-triacetin. As shown in FIG. 7, the r-triacetin formed by this process may include physical recycled content from the r-syngas and/or r-CO from the carbon reforming step. The esterification step in FIG. 7 can be performed in the presence of any esterification catalyst known in the art useful for esterifying carboxylic acids with alcohols. Examples of such catalysts include sulfuric acid, heteropolyacids, tungstophosphoric acid, solid acid catalysts, an alkyl sulfonic acid of the formula R′SO3H where R′ represents a substituted or unsubstituted aliphatic hydrocarbon group, an alkyl benzene sulfonic acid of the formula R″C6H4SO3H where R″ represents an alkyl substituent, metal oxides, metal alkoxides, metal hydroxides, and combinations thereof.

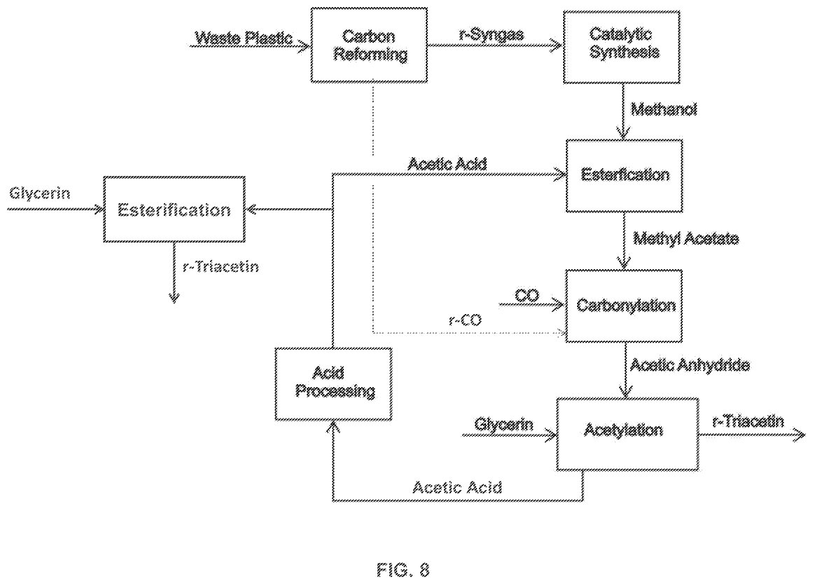

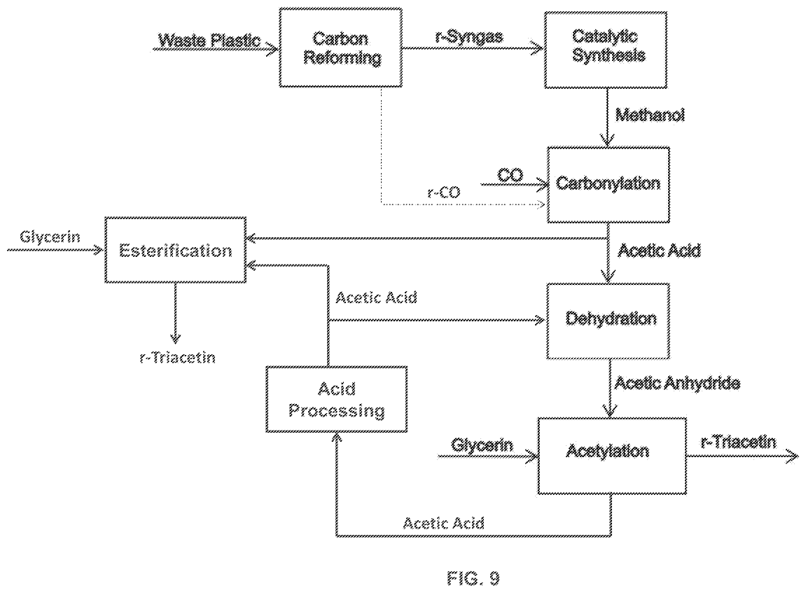

FIGS. 8 and 9 depict triacetin production processes and facilities similar to those shown in FIGS. 1 and 2, respectively. The difference in the processes shown in FIGS. 8 and 9 is that at least a portion of the acetic acid formed during the acetylation step in FIG. 1 or FIG. 2 (not shown) and/or during the carbonylation of methanol in FIG. 2 can be removed and used to esterify a glycerin in a separate step, thereby forming additional r-triacetin. As shown in FIGS. 8 and 9, at least a portion of the recycled content in the additional stream of r-triacetin formed by esterification can be physical content from the r-syngas and/or r-CO from the carbon reforming step.

In one embodiment, the glycerin reacted with the acetic acid (in FIGS. 7-9) or with the acetic anhydride (in FIGS. 1-6, 8, and 9) can be sustainable content glycerin (s-glycerin). Such glycerin may include sustainable content from one or more biological sources, such as plants (e.g., palm, soybeans, etc.) and/or animals (e.g., animal tallow). When s-glycerin is used to form triacetin as described herein, the triacetin may include both recycled content derived from waste plastic and sustainable content derived from one or more plant and/or animal sources.

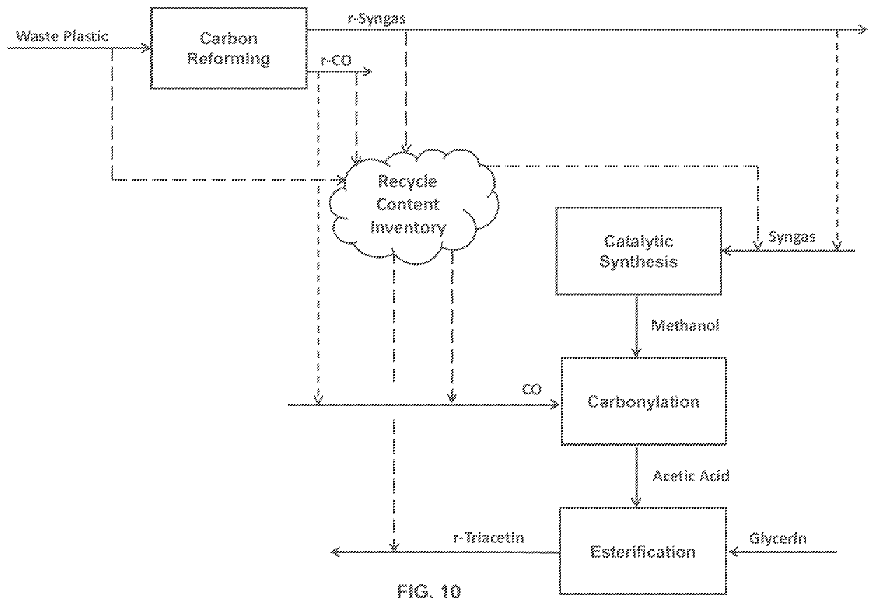

FIG. 10 shows a process and facility where the main steps (i.e., carbon reforming, catalytic synthesis, carbonylation, and esterification) are the same as the main steps shown in FIG. 7. It should be understood, however, that the unique features illustrated in FIG. 10 can also be applied to the processes shown in FIG. 8, where the main steps include carbon reforming, catalytic synthesis, esterification, carbonylation, acetylation, and esterification, and in FIG. 9, where the main steps include carbon reforming, catalytic synthesis, carbonylation, dehydration, acetylation, and esterification.

FIG. 10 illustrates a process and facility wherein the r-triacetin can include credit-based recycled content. In some cases, the r-triacetin can include credit-based recycled content from the r-syngas and may include physical, credit-based, and/or no recycled content from the r-CO (or CO). In other cases, the r-triacetin can include credit-based recycled content from the r-CO and can include physical, credit-based, and/or no recycled content from the r-syngas (or syngas). Additionally, as shown in FIG. 10, at least a portion of the recycled content from the waste plastic (or syngas or CO) may be applied directly to the triacetin product. In some cases, this may not be possible since the source material (waste plastic, syngas, and/or CO) would not have the same or similar composition as the target material (triacetin). The methods of applying recycled content credits described herein are also applicable to the system illustrated in FIG. 10.

In the embodiment depicted in FIG. 10, the r-triacetin product can have a total recycled content of 25 to 90, 40 to 80, or 55 to 65 percent if, for example, (i) the syngas fed to catalytic synthesis has 100 percent credit-based recycled content and (ii) the r-CO fed to carbonylation has 100 percent physical recycled content. In such a scenario, the r-triacetin can have both physical recycled content and credit-based recycled content, including, for example, (i) 10 to 60, 20 to 50, or 25 to 40 percent credit-based recycled content from the r-syngas and (ii) 10 to 60, 20 to 50, or 25 to 40 percent physical recycled content from the r-CO.

If glycerin having 100 percent recycled content (or sustainable content) is provided, it enables the production of r-triacetin having 100 percent recycled (or 100 percent recycled and sustainable) content, so long as the other feeds (i.e., syngas and CO) also have 100 percent recycled content. In such a scenario, the 100 percent recycled content (or 100 percent recycled and sustainable content) r-triacetin can have, for example, (i) 10 to 60, 20 to 50, or 25 to 40 percent recycled content (physical and/or credit-based) from the r-syngas, (ii) 10 to 60, 20 to 50, or 25 to 40 percent recycled content (physical and/or credit-based) from the r-CO, and (iii) 15 to 65, 25 to 55, 35 to 45 recycled content (or sustainable content, whether physical and/or credit-based) from r-glycerin (or s-glycerin).

### Claim Supporting Description—First Embodiment

In a first embodiment of the present technology there is provided a process for producing triacetin having recycled content (r-triacetin), where the process comprises the following steps: (a) carbon reforming a feed comprising waste plastic to thereby produce a recycled content syngas (r-syngas); (b) converting at least a portion of the r-syngas to recycled content methanol (r-methanol) via catalytic synthesis; (c) producing a recycled content acetic anhydride (r-acetic anhydride) from at least a portion of the r-methanol and a carbon monoxide (CO); and (d) acetylating a glycerin with at least a portion of the r-acetic anhydride to thereby provide the r-triacetin.

The first embodiment described in the preceding paragraph can also include one or more of the additional aspects listed in the following paragraphs. The each of the following additional aspects of the first embodiment can be standalone features or can be combined with one or more of the other additional aspects to the extent consistent.

In an additional aspect of the first embodiment, the r-triacetin has at least 10, 20, 30, 40 or 50 percent total recycled content.

In an additional aspect of the first embodiment, the r-triacetin has at least 10, 20, 30, 40 or 50 percent physical recycled content and less than 20, 10, 5, 1 percent credit-based recycled content.

In an additional aspect of the first embodiment, the process further comprises obtaining certification from a certification entity for the amount of recycled content in the r-triacetin.

In an additional aspect of the first embodiment, the producing of step (c) comprises esterifying an acetic acid with at least a portion of the methanol to thereby produce a methyl acetate and then carbonylating at least a portion of the methyl acetate with the CO to produce the acetic anhydride.

In an additional aspect of the first embodiment, the producing of step (c) comprises carbonylating at least a portion of the methanol with the CO to produce an acetic acid and dehydrating at least a portion of the acetic acid to produce the acetic anhydride.

In an additional aspect of the first embodiment, the process further comprises tracing recycled content along a chemical pathway from the waste plastic to the triacetin.

In an additional aspect of the first embodiment, the tracing includes determining one or more conversion factors for one or more chemical reactions along the chemical pathway, wherein the conversion factors determine how much of the physical recycled content from the waste plastic is allocated to the triacetin.

### Claim Supporting Description—Second Embodiment

In a second embodiment of the present technology there is provided a process for producing triacetin having recycled content (r-triacetin), where the process comprises the following steps: (a) converting a syngas to a methanol via catalytic synthesis; (b) producing an acetic anhydride from at least a portion of the methanol and a carbon monoxide (CO); (c) acetylating a glycerin with at least a portion of the acetic anhydride to thereby provide a triacetin; and (d) applying recycled content to at least a portion of the triacetin to thereby provide a recycled content triacetin (r-triacetin). The applying of step (d) includes (i) attributing recycled content from at least one source material having physical recycled content to at least one target material via recycled content credits, (ii) tracing recycled content along at least one chemical pathway from the at least one target material to the triacetin, and (iii) allocating recycled content to the triacetin based at least in part on the tracing of recycled content along the chemical pathway.

The second embodiment described in the preceding paragraph can also include one or more of the additional aspects listed in the following paragraphs. The each of the following additional aspects of the second embodiment can be standalone features or can be combined with one or more of the other additional aspects to the extent consistent.

In an additional aspect of the second embodiment, the applying of step (d) uses mass balance.

In an additional aspect of the second embodiment, the process further comprises obtaining certification from a certification entity for the applying of step (d).

In an additional aspect of the second embodiment, the recycled content of the source material is from waste plastic.

In an additional aspect of the second embodiment, the source material and the target material comprise the same type of material.

In an additional aspect of the second embodiment, at least one of the following criterial is met (i) the source material and the target material both comprise syngas, (ii) the source material and the target material both comprise carbon monoxide, and/or (iii) the source material and the target material both comprise glycerin.

In an additional aspect of the second embodiment, the source material and the target material have substantially the same physical composition.

In an additional aspect of the second embodiment, at least 50, 75, 90, 95, 99, or 100 weight percent of the source material is identical to the target material.

In an additional aspect of the second embodiment, the attributing includes (i) booking recycled content credits attributable to the at least one source material into a digital inventory and (ii) assigning recycled content credits from the digital inventory to the target material.

In an additional aspect of the second embodiment, the tracing includes determining one or more conversion factors for one or more chemical reactions along the chemical pathway.

In an additional aspect of the second embodiment, the conversion factors account for the conversion, yield, and selectivity of the chemical reactions in the chemical pathway.

In an additional aspect of the second embodiment, the attributing includes assigning credit-based recycled content from a digital inventory to the target material and the conversion factors determine how much of the credit-based recycled content applied to the target material is allocated to the triacetin.

In an additional aspect of the second embodiment, the r-triacetin has a total recycled content of at least 10, 20, 30, 40, 50, 75, 90, 95, or 100 percent.

In an additional aspect of the second embodiment, the r-triacetin has both physical recycled content and credit-based recycled content.

In an additional aspect of the second embodiment, the r-triacetin has at least 10, 20, 30, 40, 50 percent physical recycled content and at least 10, 20, 30, 40, or 50 percent credit-based recycled content.

In an additional aspect of the second embodiment, the source material comprises a recycled content syngas (r-syngas) having physical recycled content and the target material comprises the syngas of step (a).

In an additional aspect of the second embodiment, the r-triacetin has 10 to 60, 20 to 50, or 25 to 40 percent credit-based recycled content from the r-syngas.

In an additional aspect of the second embodiment, the source material comprises a recycled content carbon monoxide (r-CO) having physical recycled content and the target material comprises the CO of step (b).

In an additional aspect of the second embodiment, the r-triacetin has 10 to 60, 20 to 50, or 25 to 40 percent credit-based recycled content from r-CO.

In an additional aspect of the second embodiment, the source material comprises a recycled content glycerin (r-glycerin) having physical recycled content and the target material comprises the glycerin of step (c).

In an additional aspect of the second embodiment, the r-triacetin has 15 to 65, 25 to 55, 35 to 45 percent credit-based recycled content from the r-glycerin.

In an additional aspect of the second embodiment, the source material has physical recycled content and the target material has less than 100 percent physical recycled content.

In an additional aspect of the second embodiment, the source material has at least 10, 25, 50, 75, 90, or 99 percent physical recycled content.

In an additional aspect of the second embodiment, the target material has less than 99, 90, 75, 50, 25, 10, or 1 percent physical recycled content.

In an additional aspect of the second embodiment, the source material has 100 percent physical recycled content and the target material has no physical recycled content.

In an additional aspect of the second embodiment, none of the syngas, the CO, and the glycerin have physical recycled content.

In an additional aspect of the second embodiment, at least one of the syngas, the CO, and the glycerin has physical recycled content.

In an additional aspect of the second embodiment, at least two of the syngas, the CO, and the glycerin have physical recycled content.

In an additional aspect of the second embodiment, all three of the syngas, the CO, and the glycerin have physical recycled content.

In an additional aspect of the second embodiment, the applying further comprises applying physical recycled content to at least a portion of the triacetin so that the r-triacetin has both physical recycled content and credit-based recycled content.

In an additional aspect of the second embodiment, the physical recycled content applied to the triacetin is from at least one of the syngas, the CO, and the glycerin.

In an additional aspect of the second embodiment, the target material comprises the syngas and at least a portion of the credit-based recycled content allocated to the triacetin is traced through a first chemical pathway from the syngas to the triacetin.

In an additional aspect of the second embodiment, at least a portion of the physical recycled content applied to the triacetin is from the CO and is traced through a second chemical pathway from the CO to the triacetin.

In an additional aspect of the second embodiment, the source material comprises at least one of (i) a waste plastic, (ii) a recycled content syngas (r-syngas) having physical recycled content, (iii) a recycled content carbon monoxide (r-CO) having physical recycled content, and/or (iv) a recycled content glycerin (r-glycerin) having physical recycled content.

In an additional aspect of the second embodiment, the target material comprises at least one of (i) the syngas, (ii) the CO, and/or (iii) the glycerin.

In an additional aspect of the second embodiment, the r-triacetin has credit-based recycled content from the r-syngas.

In an additional aspect of the second embodiment, the r-triacetin has physical recycled content from at least one of the r-CO and/or the r-glycerin.

In an additional aspect of the second embodiment, the source material comprises the r-syngas.

In an additional aspect of the second embodiment, the applying includes attributing credit-based recycled content from the r-syngas to the syngas via a digital inventory.

In an additional aspect of the second embodiment, the chemical pathway includes the catalytic synthesis, carbonylation with the CO, and the acetylation.

In an additional aspect of the second embodiment, the process further comprises producing the r-syngas by carbon reforming a feed comprising a recycled content feed component.

In an additional aspect of the second embodiment, the recycled content feed component comprises waste plastic and/or a material obtained from waste plastic.

In an additional aspect of the second embodiment, the feed to the carbon reforming further comprises a non-recycled feed component.

In an additional aspect of the second embodiment, the non-recycled feed component comprises at least one of coal, a liquid hydrocarbon, and a gaseous hydrocarbon.

In an additional aspect of the second embodiment, the carbon reforming comprises partial oxidation gasification.

In an additional aspect of the second embodiment, at least a portion of the r-syngas is produced at a first site and the triacetin is produced at a second site.

In an additional aspect of the second embodiment, the first and second sites are space from one another by at least 0.1, 0.5, 1, 5, 10, 50, 100, 500, or 1000 miles.

In an additional aspect of the second embodiment, at least a portion of the r-syngas is produced by a different entity than the entity producing the triacetin.

In an additional aspect of the second embodiment, the source material comprises the r-CO.

In an additional aspect of the second embodiment, the applying includes attributing credit-based recycled content from the r-CO to the CO via a digital inventory.

In an additional aspect of the second embodiment, the chemical pathway includes a carbonylation fed with the CO and the acetylation.

In an additional aspect of the second embodiment, the process further comprises producing the r-CO by carbon reforming a feed comprising a recycled content feed component.

In an additional aspect of the second embodiment, the recycled content feed component comprises waste plastic and/or a material obtained from waste plastic.

In an additional aspect of the second embodiment, at least a portion of the r-CO is produced via gasification of a feed comprising waste plastic.

In an additional aspect of the second embodiment, at least a portion of the r-CO is produced at a first site and the triacetin is produced at a second site.

In an additional aspect of the second embodiment, the first and second sites are space from one another by at least 0.1, 0.5, 1, 5, 10, 50, 100, 500, or 1000 miles.

In an additional aspect of the second embodiment, at least a portion of the r-CO is produced by a different entity than the entity producing the triacetin.

In an additional aspect of the second embodiment, the source material comprises the r-glycerin.

In an additional aspect of the second embodiment, the applying includes attributing credit-based recycled content from the r-glycerin to the glycerin via a digital inventory.

In an additional aspect of the second embodiment, the chemical pathway includes the acetylation.

In an additional aspect of the second embodiment, at least a portion of the r-glycerin is produced at a first site and the triacetin is produced at a second site.

In an additional aspect of the second embodiment, the first and second sites are space from one another by at least 0.1, 0.5, 1, 5, 10, 50, 100, 500, or 1000 miles.

In an additional aspect of the second embodiment, at least a portion of the r-glycerin is produced by a different entity than the entity producing the triacetin.

In an additional aspect of the second embodiment, the r-triacetin has a total recycled content of 100 percent.

In an additional aspect of the second embodiment, a first portion of the total recycled content is attributable to a recycled content syngas (r-syngas) having physical recycled content, a second portion of the total recycled content is attributable to a recycled content CO (r-CO) having physical recycled content, and a third portion of the total recycled content is attributable to a recycled content glycerin (r-glycerin) having physical recycled content.

In an additional aspect of the second embodiment, the first portion of the total recycled content is 10 to 50 percent, the second portion of the total recycled content is 10 to 50 percent, and the third portion of the total recycled content is 10 to 50 percent.

### Claim Supporting Description—Third Embodiment

In a third embodiment of the present technology there is provided a process for producing triacetin having recycled content (r-triacetin), the process comprising: (a) carbon reforming a feed comprising waste plastic to thereby produce a recycled content syngas (r-syngas); (b) converting at least a portion of the r-syngas to recycled content methanol (r-methanol) via catalytic synthesis; (c) carbonylating at least a portion of the r-methanol with a carbon monoxide (CO) to form recycled content acetic acid (r-acetic acid); and (d) esterifying a glycerin with at least a portion of the r-acetic acid to thereby provide the r-triacetin.

The third embodiment described in the preceding paragraph can also include one or more of the additional aspects listed in the following paragraphs. The each of the following additional aspects of the third embodiment can be standalone features or can be combined with one or more of the other additional aspects to the extent consistent.

In an additional aspect of the third embodiment, the r-triacetin has at least 10, 20, 30, 40 or 50 percent total recycled content.

In an additional aspect of the third embodiment, the r-triacetin has at least 10, 20, 30, 40 or 50 percent physical recycled content and less than 20, 10, 5, 1 percent credit-based recycled content.

In an additional aspect of the third embodiment, the process further comprises obtaining certification from a certification entity for the amount of recycled content in the r-triacetin.

In an additional aspect of the third embodiment, the process further comprises tracing recycled content along a chemical pathway from the waste plastic to the triacetin.

In an additional aspect of the third embodiment, the tracing includes determining one or more conversion factors for one or more chemical reactions along the chemical pathway, wherein the conversion factors determine how much of the physical recycled content from the waste plastic is allocated to the triacetin.

In an additional aspect of the third embodiment, at least a portion of the CO comprises recycled content CO (r-CO).

In an additional aspect of the third embodiment, further comprising dehydrating at least a portion of the r-acetic acid to provide recycled content acetic anhydride (r-anhydride).

In an additional aspect of the third embodiment, the esterifying is performed in the presence of at least one esterification catalyst including but not limited to at least one selected from the group consisting of sulfuric acid, heteropolyacids, tungstophosphoric acid, solid acid catalysts, an alkyl sulfonic acid of the formula R′SO3H where R′ represents a substituted or unsubstituted aliphatic hydrocarbon group, an alkyl benzene sulfonic acid of the formula R″C6H4SO3H where R″ represents an alkyl substituent, metal oxides, metal alkoxides, metal hydroxides, and combinations thereof.

In an additional aspect of the third embodiment, at least a portion of the glycerin comprises sustainable content glycerin (s-glycerin) from at least one sustainable source.

In an additional aspect of the third embodiment, tracing recycled content along a chemical pathway from the waste plastic to the triacetin, wherein the tracing includes determining one or more conversion factors for one or more chemical reactions along the chemical pathway, and wherein the conversion factors determine how much of the physical recycled content from the waste plastic is allocated to the triacetin.

### Claim Supporting Description—Fourth Embodiment

In a fourth embodiment of the present technology there is provided a process for producing triacetin having recycled content (r-triacetin), the process comprising: (a) carbon reforming a feed comprising waste plastic to thereby produce a recycled content syngas (r-syngas); (b) converting at least a portion of the r-syngas to recycled content methanol (r-methanol) via catalytic synthesis; (c) producing a recycled content acetic anhydride (r-acetic anhydride) from at least a portion of the r-methanol and a carbon monoxide (CO); (d) recovering a recycled content acetic acid (r-acetic acid) from an acetylation with at least a portion of the r-acetic anhydride; and (e) esterifying a glycerin with at least a portion of the r-acetic acid to thereby provide the r-triacetin.

The fourth embodiment described in the preceding paragraph can also include one or more of the additional aspects listed in the following paragraphs. The each of the following additional aspects of the fourth embodiment can be standalone features or can be combined with one or more of the other additional aspects to the extent consistent.

In an additional aspect of the fourth embodiment, the r-triacetin has at least 10, 20, 30, 40 or 50 percent total recycled content.

In an additional aspect of the fourth embodiment, the r-triacetin has at least 10, 20, 30, 40 or 50 percent physical recycled content and less than 20, 10, 5, 1 percent credit-based recycled content.

In an additional aspect of the fourth embodiment, the process further comprises obtaining certification from a certification entity for the amount of recycled content in the r-triacetin.

In an additional aspect of the fourth embodiment, the producing of step (c) comprises carbonylating at least a portion of the methanol with the CO to produce an acetic acid and dehydrating at least a portion of the acetic acid to produce the acetic anhydride.

In an additional aspect of the fourth embodiment, the process further comprises tracing recycled content along a chemical pathway from the waste plastic to the triacetin.

In an additional aspect of the fourth embodiment, the tracing includes determining one or more conversion factors for one or more chemical reactions along the chemical pathway, wherein the conversion factors determine how much of the physical recycled content from the waste plastic is allocated to the triacetin.

In an additional aspect of the fourth embodiment, the acetylation comprises acetylating glycerin with at least a portion of the r-acetic anhydride to form additional recycled content triacetin (r-triacetin).

In an additional aspect of the fourth embodiment, at least a portion of the glycerin esterified in step (c) comprises sustainable content glycerin (s-glycerin).

In an additional aspect of the fourth embodiment, the esterifying of step (e) is performed in the presence of an esterification catalyst including but not limited to at least one selected from the group consisting of sulfuric acid, heteropolyacids, tungstophosphoric acid, solid acid catalysts, an alkyl sulfonic acid of the formula R′SO3H where R′ represents a substituted or unsubstituted aliphatic hydrocarbon group, an alkyl benzene sulfonic acid of the formula R″C6H4SO3H where R″ represents an alkyl substituent, metal oxides, metal alkoxides, metal hydroxides, and combinations thereof.

### Claim Supporting Description—Fifth Embodiment

In a fifth embodiment of the present technology there is provided a process for producing triacetin having recycled content (r-triacetin), the process comprising: (a) converting a syngas to a methanol via catalytic synthesis; (b) producing an acetic acid from at least a portion of the methanol and a carbon monoxide (CO); (c) esterifying a glycerin with at least a portion of the acetic acid to thereby provide a triacetin; and (d) applying recycled content to at least a portion of the triacetin to thereby provide the r-triacetin, wherein the applying of step (d) includes (i) attributing recycled content from at least one source material having physical recycled content to at least one target material via recycled content credits, (ii) tracing recycled content along at least one chemical pathway from the at least one target material to the triacetin, and (iii) allocating recycled content to the triacetin based at least in part on the tracing of recycled content along the chemical pathway.

The fifth embodiment described in the preceding paragraph can also include one or more of the additional aspects listed in the following paragraphs. The each of the following additional aspects of the fifth embodiment can be standalone features or can be combined with one or more of the other additional aspects to the extent consistent.

In an additional aspect of the fifth embodiment, the applying of step (d) uses mass balance.

In an additional aspect of the fifth embodiment, the process further comprises obtaining certification from a certification entity for the applying of step (d).

In an additional aspect of the fifth embodiment, the recycled content of the source material is from waste plastic.

In an additional aspect of the fifth embodiment, the source material and the target material comprise the same type of material.

In an additional aspect of the fifth embodiment, at least one of the following criterial is met (i) the source material and the target material both comprise syngas, (ii) the source material and the target material both comprise carbon monoxide, and/or (iii) the source material and the target material both comprise glycerin.

In an additional aspect of the fifth embodiment, the source material and the target material have substantially the same physical composition.

In an additional aspect of the fifth embodiment, at least 50, 75, 90, 95, 99, or 100 weight percent of the source material is identical to the target material.

In an additional aspect of the fifth embodiment, the attributing includes (i) booking recycled content credits attributable to the at least one source material into a digital inventory and (ii) assigning recycled content credits from the digital inventory to the target material.

In an additional aspect of the fifth embodiment, the tracing includes determining one or more conversion factors for one or more chemical reactions along the chemical pathway.

In an additional aspect of the fifth embodiment, the conversion factors account for the conversion, yield, and selectivity of the chemical reactions in the chemical pathway.

In an additional aspect of the fifth embodiment, the attributing includes assigning credit-based recycled content from a digital inventory to the target material and the conversion factors determine how much of the credit-based recycled content applied to the target material is allocated to the triacetin.

In an additional aspect of the fifth embodiment, the r-triacetin has a total recycled content of at least 10, 20, 30, 40, 50, 75, 90, 95, or 100 percent.

In an additional aspect of the fifth embodiment, the r-triacetin has both physical recycled content and credit-based recycled content.

In an additional aspect of the fifth embodiment, the r-triacetin has at least 10, 20, 30, 40, 50 percent physical recycled content and at least 10, 20, 30, 40, or 50 percent credit-based recycled content.

In an additional aspect of the fifth embodiment, the source material comprises a recycled content syngas (r-syngas) having physical recycled content and the target material comprises the syngas of step (a).

In an additional aspect of the fifth embodiment, the r-triacetin has 10 to 60, 20 to 50, or 25 to 40 percent credit-based recycled content from the r-syngas.

In an additional aspect of the fifth embodiment, the source material comprises a recycled content carbon monoxide (r-CO) having physical recycled content and the target material comprises the CO of step (b).

In an additional aspect of the fifth embodiment, the r-triacetin has 10 to 60, 20 to 50, or 25 to 40 percent credit-based recycled content from r-CO.

In an additional aspect of the fifth embodiment, the source material comprises a recycled content glycerin (r-glycerin) having physical recycled content and the target material comprises the glycerin of step (c).

In an additional aspect of the fifth embodiment, the r-triacetin has 15 to 65, 25 to 55, 35 to 45 percent credit-based recycled content from the r-glycerin.

In an additional aspect of the fifth embodiment, the source material comprises a sustainable content glycerin (s-glycerin) having physical recycled content and the target material comprises the glycerin of step (c).

In an additional aspect of the fifth embodiment, the r-triacetin has 15 to 65, 25 to 55, 35 to 45 percent credit-based sustainable content from the s-glycerin.

In an additional aspect of the fifth embodiment, the source material has physical recycled content and the target material has less than 100 percent physical recycled content.

In an additional aspect of the fifth embodiment, the source material has at least 10, 25, 50, 75, 90, or 99 percent physical recycled content.

In an additional aspect of the fifth embodiment, the target material has less than 99, 90, 75, 50, 25, 10, or 1 percent physical recycled content.

In an additional aspect of the fifth embodiment, the source material has 100 percent physical recycled content and the target material has no physical recycled content.

In an additional aspect of the fifth embodiment, none of the syngas, the CO, and the glycerin have physical recycled content.

In an additional aspect of the fifth embodiment, at least one of the syngas, the CO, and the glycerin has physical recycled content.

In an additional aspect of the fifth embodiment, at least two of the syngas, the CO, and the glycerin have physical recycled content.

In an additional aspect of the fifth embodiment, all three of the syngas, the CO, and the glycerin have physical recycled content.

In an additional aspect of the fifth embodiment, the applying further comprises applying physical recycled content to at least a portion of the triacetin so that the r-triacetin has both physical recycled content and credit-based recycled content.

In an additional aspect of the fifth embodiment, the physical recycled content applied to the triacetin is from at least one of the syngas, the CO, and the glycerin.

In an additional aspect of the fifth embodiment, the target material comprises the syngas and at least a portion of the credit-based recycled content allocated to the triacetin is traced through a fifth chemical pathway from the syngas to the triacetin.

In an additional aspect of the fifth embodiment, at least a portion of the physical recycled content applied to the triacetin is from the CO and is traced through a fifth chemical pathway from the CO to the triacetin.

In an additional aspect of the fifth embodiment, the source material comprises at least one of (i) a waste plastic, (ii) a recycled content syngas (r-syngas) having physical recycled content, (iii) a recycled content carbon monoxide (r-CO) having physical recycled content, and/or (iv) a recycled content glycerin (r-glycerin) having physical recycled content.

In an additional aspect of the fifth embodiment, the target material comprises at least one of (i) the syngas, (ii) the CO, and/or (iii) the glycerin.

In an additional aspect of the fifth embodiment, the r-triacetin has credit-based recycled content from the r-syngas.

In an additional aspect of the fifth embodiment, the r-triacetin has physical recycled content from at least one of the r-CO and/or the r-glycerin.

In an additional aspect of the fifth embodiment, the source material comprises the r-syngas.

In an additional aspect of the fifth embodiment, the applying includes attributing credit-based recycled content from the r-syngas to the syngas via a digital inventory.

In an additional aspect of the fifth embodiment, the chemical pathway includes the catalytic synthesis, carbonylation with the CO, and the acetylation.

In an additional aspect of the fifth embodiment, the process further comprises producing the r-syngas by carbon reforming a feed comprising a recycled content feed component.

In an additional aspect of the fifth embodiment, the recycled content feed component comprises waste plastic and/or a material obtained from waste plastic.

In an additional aspect of the fifth embodiment, the feed to the carbon reforming further comprises a non-recycled feed component.

In an additional aspect of the fifth embodiment, the non-recycled feed component comprises at least one of coal, a liquid hydrocarbon, and a gaseous hydrocarbon.

In an additional aspect of the fifth embodiment, the carbon reforming comprises partial oxidation gasification.

In an additional aspect of the fifth embodiment, at least a portion of the r-syngas is produced at a fifth site and the triacetin is produced at a fifth site.

In an additional aspect of the fifth embodiment, the fifth and fifth sites are space from one another by at least 0.1, 0.5, 1, 5, 10, 50, 100, 500, or 1000 miles.

In an additional aspect of the fifth embodiment, at least a portion of the r-syngas is produced by a different entity than the entity producing the triacetin.

In an additional aspect of the fifth embodiment, the source material comprises the r-CO.

In an additional aspect of the fifth embodiment, the applying includes attributing credit-based recycled content from the r-CO to the CO via a digital inventory.

In an additional aspect of the fifth embodiment, the chemical pathway includes a carbonylation fed with the CO and the acetylation.

In an additional aspect of the fifth embodiment, the process further comprises producing the r-CO by carbon reforming a feed comprising a recycled content feed component.

In an additional aspect of the fifth embodiment, the recycled content feed component comprises waste plastic and/or a material obtained from waste plastic.

In an additional aspect of the fifth embodiment, at least a portion of the r-CO is produced via gasification of a feed comprising waste plastic.

In an additional aspect of the fifth embodiment, at least a portion of the r-CO is produced at a fifth site and the triacetin is produced at a fifth site.

In an additional aspect of the fifth embodiment, the fifth and fifth sites are space from one another by at least 0.1, 0.5, 1, 5, 10, 50, 100, 500, or 1000 miles.

In an additional aspect of the fifth embodiment, at least a portion of the r-CO is produced by a different entity than the entity producing the triacetin.

In an additional aspect of the fifth embodiment, the source material comprises the r-glycerin.

In an additional aspect of the fifth embodiment, the applying includes attributing credit-based recycled content from the r-glycerin to the glycerin via a digital inventory.

In an additional aspect of the fifth embodiment, the chemical pathway includes the acetylation.

In an additional aspect of the fifth embodiment, at least a portion of the r-glycerin is produced at a fifth site and the triacetin is produced at a fifth site.

In an additional aspect of the fifth embodiment, the fifth and fifth sites are space from one another by at least 0.1, 0.5, 1, 5, 10, 50, 100, 500, or 1000 miles.

In an additional aspect of the fifth embodiment, at least a portion of the r-glycerin is produced by a different entity than the entity producing the triacetin.

In an additional aspect of the fifth embodiment, the r-triacetin has a total recycled content of 100 percent.

In an additional aspect of the fifth embodiment, a fifth portion of the total recycled content is attributable to a recycled content syngas (r-syngas) having physical recycled content, a fifth portion of the total recycled content is attributable to a recycled content CO (r-CO) having physical recycled content, and a third portion of the total recycled content is attributable to a recycled content glycerin (r-glycerin) having physical recycled content.

In an additional aspect of the fifth embodiment, the fifth portion of the total recycled content is 10 to 50 percent, the fifth portion of the total recycled content is 10 to 50 percent, and the third portion of the total recycled content is 10 to 50 percent.

### Definitions

It should be understood that the following is not intended to be an exclusive list of defined terms. Other definitions may be provided in the foregoing description, such as, for example, when accompanying the use of a defined term in context.

As used herein, the terms “a,” “an,” and “the” mean one or more.

As used herein, the term “and/or,” when used in a list of two or more items, means that any one of the listed items can be employed by itself or any combination of two or more of the listed items can be employed. For example, if a composition is described as containing components A, B, and/or C, the composition can contain A alone; B alone; C alone; A and B in combination; A and C in combination, B and C in combination; or A, B, and C in combination.

As used herein, the phrase “at least a portion” includes at least a portion and up to and including the entire amount or time period.

As used herein, the term “chemical pathway” refers to the chemical processing step or steps (e.g., chemical reactions, physical separations, etc.) between an input material and a product material, where the input material is used to make the product material.

As used herein, the term “chemical recycling” refers to a waste plastic recycling process that includes a step of chemically converting waste plastic polymers into lower molecular weight polymers, oligomers, monomers, and/or non-polymeric molecules (e.g., hydrogen, carbon monoxide, methane, ethane, propane, ethylene, and CO) that are useful by themselves and/or are useful as feedstocks to another chemical production process(es).

As used herein, the term “co-located” refers to the characteristic of at least two objects being situated on a common physical site, and/or within one mile of each other.

As used herein, the terms “comprising,” “comprises,” and “comprise” are open-ended transition terms used to transition from a subject recited before the term to one or more elements recited after the term, where the element or elements listed after the transition term are not necessarily the only elements that make up the subject.

As used herein, the terms “credit-based recycled content,” “non-physical recycled content,” and “indirect recycled content” all refer to matter that is not physically traceable back to a waste material, but to which a recycled content credit has been attributed.

As used herein, the term “directly derived” refers to having at least one physical component originating from waste material.

As used herein, the terms “including,” “include,” and “included” have the same open-ended meaning as “comprising,” “comprises,” and “comprise” provided above.

As used herein, the term “indirectly derived” refers to having an applied recycled content (i) that is attributable to waste material, but (ii) that is not based on having a physical component originating from waste material.

As used herein, the term “located remotely” refers to a distance of at least 0.1, 0.5, 1, 5, 10, 50, 100, 500, or 1000 miles between two facilities, sites, or reactors.

As used herein, the term “mass balance” refers to a method of tracking recycled content based on the mass of the recycled content in various materials.

As used herein, the terms “physical recycled content” and “direct recycled content” both refer to matter that is physically traceable back to a waste material.

As used herein, the term “predominantly” means more than 50 percent by weight. For example, a predominantly propane stream, composition, feedstock, or product is a stream, composition, feedstock, or product that contains more than 50 weight percent propane.

As used herein, the term “recycled content” refers to being or comprising a composition that is directly and/or indirectly derived from recycle material. Recycled content is used generically to refer to both physical recycled content and credit-based recycled content. Recycled content is also used as an adjective to describe material having physical recycled content and/or credit-based recycled content.

As used herein, the term “recycled content credit” refers to a non-physical measure of physical recycled content that can be directly or indirectly (i.e., via a digital inventory) attributed from a first material having physical recycled content to a second material having less than 100 percent physical recycled content.

As used herein, the term “total recycled content” refers to the cumulative amount of physical recycled content and credit-based recycled content from all sources.

As used herein, the term “waste material” refers to used, scrap, and/or discarded material.

As used herein, the terms “waste plastic” and “plastic waste” refer to used, scrap, and/or discarded plastic materials.

As used herein, the term “esterification catalyst” refers to any compound with properties sufficient to catalyze an esterification reaction. Examples include, but are not limited to, strongly acidic molecules, sulfuric acid, heteropolyacids, tungstophosphoric acid, solid acid catalysts, an alkyl sulfonic acid of the formula R′SO3H where R′ represents a substituted or unsubstituted aliphatic hydrocarbon group, an alkyl benzene sulfonic acid of the formula R″C6H4SO3H where R″ represents an alkyl substituent, metal oxides, metal alkoxides, metal hydroxides, and combinations thereof.

### CLAIMS NOT LIMITED TO DISCLOSED EMBODIMENTS

The preferred forms of the invention described above are to be used as illustration only and should not be used in a limiting sense to interpret the scope of the present invention. Modifications to the exemplary embodiments, set forth above, could be readily made by those skilled in the art without departing from the spirit of the present invention.

The inventors hereby state their intent to rely on the Doctrine of Equivalents to determine and assess the reasonably fair scope of the present invention as it pertains to any apparatus not materially departing from but outside the literal scope of the invention as set forth in the following claims.

1. A process for producing triacetin having recycled content (r-triacetin), the process comprising:
(a) carbon reforming a feed comprising waste plastic to thereby produce a recycled content syngas (r-syngas);
(b) converting at least a portion of the r-syngas to recycled content methanol (r-methanol) via catalytic synthesis;
(c) producing a recycled content acetic anhydride (r-acetic anhydride) from at least a portion of the r-methanol and a carbon monoxide (CO); and
(d) acetylating a glycerin with at least a portion of the r-acetic anhydride to thereby provide the r-triacetin.

2. The process of claim 1, wherein the r-triacetin has at least 20 percent total recycled content.

3. The process of claim 1, wherein the r-triacetin has at least 10 percent physical recycled content and less than 1 percent credit-based recycled content.

4. The process of claim 1, further comprising obtaining certification from a certification entity for the amount of recycled content in the r-triacetin.

5. The process of claim 1, wherein the producing of step (c) comprises esterifying an acetic acid with at least a portion of the methanol to thereby produce a methyl acetate and then carbonylating at least a portion of the methyl acetate with the CO to produce the acetic anhydride.

6. The process of claim 1, wherein the producing of step (c) comprises carbonylating at least a portion of the methanol with the CO to produce an acetic acid and dehydrating at least a portion of the acetic acid to produce the acetic anhydride.

7. The process of claim 1, further comprising tracing recycled content along a chemical pathway from the waste plastic to the triacetin.

8. The process of claim 7, wherein the tracing includes determining one or more conversion factors for one or more chemical reactions along the chemical pathway, wherein the conversion factors determine how much of the physical recycled content from the waste plastic is allocated to the triacetin.

9. A process for producing triacetin having recycled content (r-triacetin), the process comprising:
(a) converting a syngas to a methanol via catalytic synthesis;
(b) producing an acetic anhydride from at least a portion of the methanol and a carbon monoxide (CO);
(c) acetylating a glycerin with at least a portion of the acetic anhydride to thereby provide a triacetin; and
(d) applying recycled content to at least a portion of the triacetin to thereby provide the r-triacetin,
wherein the applying of step (d) includes (i) attributing recycled content from at least one source material having physical recycled content to at least one target material via recycled content credits, (ii) tracing recycled content along at least one chemical pathway from the at least one target material to the triacetin, and (iii) allocating recycled content to the triacetin based at least in part on the tracing of recycled content along the chemical pathway.

10. The process of claim 9, wherein the applying of step (d) uses mass balance.

11. The process of claim 9, further comprising obtaining certification from a certification entity for the applying of step (d).

12. The process of claim 9, wherein the recycled content of the source material is from waste plastic.

13. The process of claim 9, wherein at least one of the following criterial is met (i) the source material and the target material both comprise syngas and/or (ii) the source material and the target material both comprise carbon monoxide.

14. The process of claim 9, wherein the attributing includes (i) booking recycled content credits attributable to the at least one source material into a digital inventory and (ii) assigning recycled content credits from the digital inventory to the target material.

15. The process of claim 9, wherein the tracing includes determining one or more conversion factors for one or more chemical reactions along the chemical pathway,
wherein the attributing includes assigning credit-based recycled content from a digital inventory to the target material,
wherein the conversion factors determine how much of the credit-based recycled content applied to the target material is allocated to the triacetin.

16. The process of claim 9, wherein the r-triacetin has a total recycled content of at least 20 percent.

17. The process of claim 9, wherein the applying further comprises applying physical recycled content to at least a portion of the triacetin so that the r-triacetin has both physical recycled content and credit-based recycled content, wherein the physical recycled content applied to the triacetin is from at least one of the syngas and the CO.

18. The process of claim 9, wherein the source material comprises at least one of (i) a waste plastic, (ii) a recycled content syngas (r-syngas) having physical recycled content, (iii) a recycled content carbon monoxide (r-CO) having physical recycled content, and/or (iv) a recycled content glycerin having physical recycled content, wherein the target material comprises at least one of (i) the syngas, (ii) the CO, and/or (iii) the glycerin.

19. The process of claim 18, wherein the source material comprises the r-syngas, wherein the applying includes attributing credit-based recycled content from the r-syngas to the syngas via a digital inventory, wherein the chemical pathway includes the catalytic synthesis, carbonylation, and the acetylation.

20. The process of claim 19, wherein at least a portion of the r-syngas is produced at a first site and the triacetin is produced at a second site, wherein the first and second sites are space from one another by at least 1 miles.

21. A process for producing triacetin having recycled content (r-triacetin), the process comprising:
(a) carbon reforming a feed comprising waste plastic to thereby produce a recycled content syngas (r-syngas);
(b) converting at least a portion of the r-syngas to recycled content methanol (r-methanol) via catalytic synthesis;
(c) carbonylating at least a portion of the r-methanol with a carbon monoxide (CO) to form recycled content acetic acid (r-acetic acid); and
(d) esterifying a glycerin with at least a portion of the r-acetic acid to thereby provide the r-triacetin.

22. The process of claim 21, wherein the CO comprises recycled content CO (r-CO).

23. The process of claim 21, further comprising dehydrating at least a portion of the r-acetic acid to provide recycled content acetic anhydride (r-anhydride).

24. The process of claim 21, wherein the esterifying of step (d) is performed in the presence of at least one esterification catalyst selected from the group consisting of sulfuric acid, heteropolyacids, tungstophosphoric acid, solid acid catalysts, an alkyl sulfonic acid of the formula R′SO3H where R′ represents a substituted or unsubstituted aliphatic hydrocarbon group, an alkyl benzene sulfonic acid of the formula R″C6H4SO3H where R″ represents an alkyl substituent, metal oxides, metal alkoxides, metal hydroxides, and combinations thereof.

25. The process of claim 21, wherein at least a portion of the glycerin comprises sustainable content glycerin (s-glycerin) from at least one sustainable source.

26. The process of claim 21, further comprising tracing recycled content along a chemical pathway from the waste plastic to the triacetin, wherein the tracing includes determining one or more conversion factors for one or more chemical reactions along the chemical pathway, and wherein the conversion factors determine how much of the physical recycled content from the waste plastic is allocated to the triacetin.

27. A process for producing triacetin having recycled content (r-triacetin), the process comprising:
(a) carbon reforming a feed comprising waste plastic to thereby produce a recycled content syngas (r-syngas);
(b) converting at least a portion of the r-syngas to recycled content methanol (r-methanol) via catalytic synthesis;
(c) producing a recycled content acetic anhydride (r-acetic anhydride) from at least a portion of the r-methanol and a carbon monoxide (CO);
(d) recovering a recycled content acetic acid (r-acetic acid) from an acetylation with at least a portion of the r-acetic anhydride; and
(e) esterifying a glycerin with at least a portion of the r-acetic acid to thereby provide the r-triacetin.

28. The process of claim 27, wherein the acetylation comprises acetylating glycerin with at least a portion of the r-acetic anhydride to form additional recycled content triacetin (r-triacetin).

29. The process of claim 27, wherein at least a portion of the glycerin esterified in step (c) comprises sustainable content glycerin (s-glycerin).

30. The process of claim 27, wherein the esterifying of step (e) is performed in the presence of an esterification catalyst selected from the group consisting of sulfuric acid, heteropolyacids, tungstophosphoric acid, solid acid catalysts, an alkyl sulfonic acid of the formula R′SO3H where R′ represents a substituted or unsubstituted aliphatic hydrocarbon group, an alkyl benzene sulfonic acid of the formula R″C6H4SO3H where R″ represents an alkyl substituent, metal oxides, metal alkoxides, metal hydroxides, and combinations thereof.

31. A process for producing triacetin having recycled content (r-triacetin), the process comprising:
(e) converting a syngas to a methanol via catalytic synthesis;
(f) producing an acetic acid from at least a portion of the methanol and a carbon monoxide (CO);
(g) esterifying a glycerin with at least a portion of the acetic acid to thereby provide a triacetin; and
(h) applying recycled content to at least a portion of the triacetin to thereby provide the r-triacetin,
wherein the applying of step (d) includes (i) attributing recycled content from at least one source material having physical recycled content to at least one target material via recycled content credits, (ii) tracing recycled content along at least one chemical pathway from the at least one target material to the triacetin, and (iii) allocating recycled content to the triacetin based at least in part on the tracing of recycled content along the chemical pathway.

32. The process of claim 31, wherein the applying of step (d) uses mass balance.

33. The process of claim 31, wherein at least one of the following criterial is met (i) the source material and the target material both comprise syngas and/or (ii) the source material and the target material both comprise carbon monoxide.

34. The process of claim 31, wherein the attributing includes (i) booking recycled content credits attributable to the at least one source material into a digital inventory and (ii) assigning recycled content credits from the digital inventory to the target material.

35. The process of claim 31, wherein the tracing includes determining one or more conversion factors for one or more chemical reactions along the chemical pathway,
wherein the attributing includes assigning credit-based recycled content from a digital inventory to the target material,
wherein the conversion factors determine how much of the credit-based recycled content applied to the target material is allocated to the triacetin.

36. The process of claim 31, wherein the applying further comprises applying physical recycled content to at least a portion of the triacetin so that the r-triacetin has both physical recycled content and credit-based recycled content, wherein the physical recycled content applied to the triacetin is from at least one of the syngas and the CO.

37. The process of claim 31, wherein the source material comprises at least one of (i) a waste plastic, (ii) a recycled content syngas (r-syngas) having physical recycled content, (iii) a recycled content carbon monoxide (r-CO) having physical recycled content, and/or (iv) a recycled content glycerin having physical recycled content, wherein the target material comprises at least one of (i) the syngas, (ii) the CO, and/or (iii) the triacetin.

38. The process of claim 21, wherein the r-triacetin has at least 20 percent total recycled content.

39. The process of claim 21, wherein the r-triacetin has at least 10 percent physical recycled content and less than 1 percent credit-based recycled content.

40. The process of claim 21, further comprising obtaining certification from a certification entity for the amount of recycled content in the r-triacetin.
